(19) United States
(12) Patent Application Publication
(10) Pub. No.: US 2004/0097484 A1
(43) Pub. Date: May 20, 2004

(54) Once a day galantamine pharmaceutical compositions and methods of use

(76) Inventors: Marc Cantillion, San Francisco, CA (US); Ann Hsu, Hayward, CA (US); Chien-Hsuan Han, Hayward, CA (US)

Correspondence Address: FLESHNER & KIM, LLP, P.O. BOX 221200, CHANTILLY, VA, 20153, US

(21) Appl. No.: 10/293,942

(22) Filed: Nov. 14, 2002

(51) Int. Cl. A61K 31/55; A61K 9/22

(52) U.S. Cl. 514/214.03; 424/468

(57) ABSTRACT

Disclosed are once a day pharmaceutical compositions containing acetylcholinesterase inhibitors, including those with nicotinic receptor modulation such as galantamine or a pharmaceutically acceptable salt thereof Also disclosed is the use of such compositions, for example, for treating or preventing cognitive or other CNS performance impairment in a mammal, such as primary or secondary memory impairment, toxic, secondary to medical or psychiatric, Alzheimer's, vascular and other dementias, mild cognitive impairments, and other cognitive impairments, such as Attention Deficit Disorder, Fibromyalgia, Chronic Fatigue Syndrome, PTSD and Down's Syndrome. This includes behavioral efficacy, as anxiety depression apathy and agitation, in addition to neurophysiological and functional outcomes including a decrease in care givers distress.

### BACKGROUND OF THE INVENTION

1. Field of the Invention

The present invention relates to once a day pharmaceutical compositions containing acetylcholinesterase inhibitors, including those with nicotinic receptor modulation such as galantamine or a pharmaceutically acceptable salt thereof. The present invention is also directed to the use of such compositions, for example, for treating or preventing cognitive or other CNS performance impairment in a mammal, such as primary or secondary memory impairment, toxic, secondary to medical or psychiatric, Alzheimer's, vascular and other dementias, mild cognitive impairments, and other cognitive impairments, such as Attention Deficit Disorder, Fibromyalgia, Chronic Fatigue Syndrome, PTSD and Down's Syndrome. This includes behavioral efficacy, as anxiety depression apathy and agitation, in addition to neurophysiological and functional outcomes including a decrease in care givers distress. Throughout the rest of this application, memory impairment and galantamine are intended to be representative of these wider diseases and drug categories, respectively.

2. Background of the Related Art

Galantamine (4a, 5, 9, 10, 11, 12-hexahydro-3-methoxy-11-methyl-6H-benzofuro [3a, 3, 2-e][2] benzazepin-6-ol) is a well-known tertiary alkaloid acetylcholinesterase inhibitor which is selectively active at nicotinic receptor sites and has substantially no effect on muscarinic receptor sides, is capable of passing the blood-brain barrier in humans, and presents no severe side effects in therapeutically necessary dosages.

More specifically, galantamine and acid addition salts thereof have been known for many years to have anticholinesterase properties. Galantamine has been isolated form the bulbs of the Caucasian snowdrops Galantanus woronowi (see Proskurnina et al., “Alkaloids of Galanthus woronowi. II. Isolation of a new alkaloid.” Zh, Obschchei Khim. 22, 1899-1902 (1952); Chem. Abs., 47:6959 (1953)) and from the common snowdrop Galanthus nivalis. Galantamine has also been prepared by chemical synthesis (see Kametani et al., J. Chem. Soc. C., 6:1043-1047 (1971); Shimizu et al., Heterocycles 8:277-282 (1977)).

Pharmacokinetic studies have been performed (see Thomsen et al., “Selective Inhibition of Human Acetylcholinesterase by Galantamine in vitro and in vivo.” Life Sciences, 46:1553-1558 (1990); “Galantamine Hydrobromide in a Long-Term Treatment of Alzheimer's Disease.” Dementia 1:46-51 (1990)). It is believed that the excellent and surprising effect possessed by galantamine is due to its specific profile of properties, the most important of which can be summarized as follows: (i) the ability to pass through the blood-brain barrier in humans; (ii) a high selectivity for acetylcholinesterase as opposed to butyrylcholinesterase (about 50-fold when measured by the in vitro method of Thomsen et al.); (iii) a sufficient elimination half-life to provide an effective plasma concentration of at least 4 hours; (iv) a relatively low toxicity in therapeutic concentrations; and (v) the ability to be effective in doses which are sufficiently low that peripheral side effects are minimized.

Galantamine hydrobromide, being a tertiary amine and lipid soluble, is absorbed rapidly from the gut and transverses the blood-brain barrier easily. The common side effects, other than the ones related to cholinergic crisis, are either nausea or vomiting, and a slight headache. These side effects are rare, however, especially when care is taken to start medication in low doses.

Galantamine and analogues and salts thereof have been used for the treatment of Alzheimer's disease and related dementias (U.S. Pat. No. 4,663,318), mania (U.S. Pat. No. 5,336,675), schizophrenia (U.S. Pat. No. 5,663,238), chronic fatigue syndrome (U.S. Pat. No. 5,312,817) and the negative effects of benzodiazepine treatment (European Patent Application No. 0,515,301). Galantamine has also been administered via a transdermal patch system for the treatment of alcoholism European Patent Application No. 0,449,247) and nicotine dependence (International Patent Application WO 94/16708).

A number of different pharmaceutical compositions containing galantamine, or a salt or analogue thereof, have been proposed, including a fast-dissolving tablet containing spray-dried lactose monohydrate and microcrystalline cellulose in a 75:25 ratio (U.S. Pat. No. 6,099,863), a controlled release composition containing particles of galantamine coated by a release rate controlling membrane (International Patent Application WO 00/38686) and transdermal patch systems (European Patent Application No. 0,449,247; International Patent Application WO 94/16708).

When treating certain conditions, however, such as Alzheimer's disease, it is advantageous to maintain the plasma level of galantamine at or near a steady state. When employing fast-dissolving tablets, this requires administration at least twice a day with concomitant peaks and troughs in the plasma level of the active ingredient. Accordingly, there is a need for a prolonged release pharmaceutical composition containing galantamine.

The above references are incorporated by reference herein where appropriate for appropriate teachings of additional or alternative details, features and/or technical background.

### SUMMARY OF THE INVENTION

An object of the invention is to solve at least the above problems and/or disadvantages and to provide at least the advantages described hereinafter.

An object of the present invention is to solve at least the problems and/or disadvantages described above and to provide at least the advantages described hereinafter.

Accordingly, it is an object of the present invention to provide prolonged release pharmaceutical compositions, as well as methods of using such pharmaceutical compositions, containing galantamine or a pharmaceutically acceptable salt thereof. Other objects, features and advantages of the present invention will be set forth in the detailed description of preferred embodiments that follows and, in part, will be apparent from the description or may be learned by practice of the invention. These objects and advantages of the invention will be realized and attained by the compositions and methods particularly pointed out in the written description and claims hereof.

In accordance with these and other objects, a first embodiment of the present invention is directed to a prolonged release pharmaceutical composition comprising: (a) a therapeutically effective amount of galantamine or a pharmaceutically acceptable salt thereof, and (b) a pharmaceutically acceptable carrier, wherein the pharmaceutical composition provides release of at least about 5% of the galantamine or pharmaceutically acceptable salt thereof after about 2 hours, at least about 25% of the galantamine or salt thereof in about six hours and at least about 50% of the galantamine or salt thereof in about ten hours, as measured in USP buffer pH 6.8 at 37° C. using USP Dissolution Apparatus 2 at 50 rpm.

Another embodiment of the present invention is directed to a method for treating or preventing memory impairment in a mammal in need thereof comprising administering to a mammal a pharmaceutical composition comprising: (a) a therapeutically effective amount of galantamine or a pharmaceutically acceptable salt thereof; and (b) a pharmaceutically acceptable carrier, wherein the pharmaceutical composition provides release of not more than about 30% of the galantanine or pharmaceutically acceptable salt thereof in about 2 hour and at least about 80% of the galantamine or salt thereof in about ten hours as measured in USP buffer pH 6.8 at 37° C. using USP Dissolution Apparatus 2 at 50 rpm.

Additional advantages, objects and feature of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIGS. 1 and 2 are graphs shows the release profiles of galantamine hydrobromide pharmaceutical compositions according to the present invention.

### DETAILED DESCRIPTION OF PREFERRED EMBODIMENTS

A first preferred embodiment of the present invention is directed to a prolonged release pharmaceutical composition comprising: (a) a therapeutically effective amount of galantamine or a pharmaceutically acceptable salt thereof; and (b) a pharmaceutically acceptable carrier. According to this embodiment of the present invention, the pharmaceutical composition provides release of at least about 30% of the galantamine or pharmaceutically acceptable salt thereof in about 2 hour and at least about 80% of the galantamine or salt thereof in about ten hours as measured in USP buffer pH 6.8 at 37° C. using USP Dissolution Apparatus 2 at 50 rpm.

According to certain preferred embodiments of the present invention, a pharmaceutically acceptable salt of galantamine may be used as the active ingredient in the prolonged release pharmaceutical composition. Illustrative examples of suitable pharmaceutically acceptable salts include, but are not limited to, hydrobromide, hydrochloride, methylsulfate and methiodide. According to a particularly preferred embodiment of the present invention, galantamine hydrobromide is used in the prolonged release pharmaceutical composition.

According to certain preferred embodiments of the present invention, the pharmaceutical composition provides release of about 1-30% of the galantamine or pharmaceutically acceptable salt thereof after about 2 hours and about 50-100% after about ten hours as measured in USP buffer pH 6.8 at 37° C. using USP Dissolution Apparatus 2 at 50 rpm. More preferably, the pharmaceutical composition provides release of about 5-30% of the galantamine or pharmaceutically acceptable salt thereof after about 2 hour, about 20-65% after about 6 hours and about 50-100% after about ten hours as measured in USP buffer pH 6.8 at 37° C. using USP Dissolution Apparatus 2 at 50 rpm. Most preferably, the pharmaceutical composition provides release of about 10-30% of the galantamine or pharmaceutically acceptable salt thereof after about 2 hours, about 25-60% after about 6 hours and about 60-100% after about 10 hours as measured in USP buffer pH 6.8 at 37° C. using USP Dissolution Apparatus 2 at 50 rpm.

Another preferred embodiment of the present invention is directed to a prolonged release pharmaceutical composition comprising: (a) a therapeutically effective amount of galantamine or a pharmaceutically acceptable salt thereof; and (b) a pharmaceutically acceptable carrier, wherein the pharmaceutical composition provides release of at least about 8% of the galantamine or pharmaceutically acceptable salt thereof after about 2 hours, at least about 30% of the galantamine or salt thereof in about six hours, and at least about 60% of the galantamine or salt thereof in about ten hours as measured in USP buffer pH 6.8 at using USP Dissolution Apparatus 2 at 50 rpm.

Another preferred embodiment of the present invention is directed to a prolonged release pharmaceutical composition comprising: (a) a therapeutically effective amount of galantamine or a pharmaceutically acceptable salt thereof; and (b) a pharmaceutically acceptable carrier, wherein the pharmaceutical composition provides release of at least about 10% of the galantamine or pharmaceutically acceptable salt thereof after about 2 hours, at least about 35% of the galantamine or salt thereof in about six hours, and at least about 65% of the galantamine or salt thereof in about ten hours as measured in USP buffer pH 6.8 at 37° C. using USP Dissolution Apparatus 2 at 50 rpm.

According to yet another preferred embodiment, at least 10% of the galantamine in pharmaceutical compositions according to the present invention remains undissolved after 10 hours i.e. less than 90% of the active agent is dissolved at 10 hours.

In yet other preferred embodiment, the release of the active agent from the pharmaceutical compositions according to this invention will have an approximately zero order release profile, and more preferably a zero order release profile.

The terms “peak” and “trough” are well known in the art and are described, for example, in U.S. Pat. No. 6,228,398, which is herein incorporated by reference in its entirety, as though set forth in full.

According to certain preferred embodiments of the present invention, the pharmaceutically acceptable carrier comprises at least one pharmaceutically acceptable gelling agent to form a dosage form, such a matrix, or diffusion-controlled composition, which requires only once a day administration. Suitable pharmaceutically acceptable gelling agents are known and available to those skilled in the art. Illustrative examples of suitable pharmaceutically acceptable gelling agents include, but are not limited to, pharmaceutically acceptable water soluble organic gums, pharmaceutically acceptable natural clays, pharmaceutically acceptable synthetic clays and mixtures thereof. An appropriate amount of gelling agent may be determined empirically by one skilled in the art.

Preferably, the pharmaceutically acceptable water soluble organic gums may be selected from the group consisting of alginates, alkyl celluloses, hydroxyalkylcelluloses, alkyl hydroxyalkylcelluloses, carboxyalkylcelluloses, carrageenan, guar gum, agar, gum arabic, gum ghatti, gum karaya, gum tragacanth, locust bean gum, pectins, polyacrylamide, polyacrylic acid, polyethylene glycol, poly(ethylene oxide), polyvinyl alcohol, polyvinylpyrrolidone, starch, tamarind gum, xanthum gum and mixtures thereof More preferably, the pharmaceutically acceptable water soluble organic gum is a mixture of xanthum gum and locust bean gum.

In alternative embodiments of the present invention, the pharmaceutically acceptable natural clays may be preferably selected from the group consisting of kaolins, serpentines, smectites (montmorillonites), bentonites, illites, glauconite, chlorites, vermiculites, mixed-layer clays, attapulgite, saponite, sepiolite and mixtures thereof. Similarly, pharmaceutically acceptable synthetic clays may be selected from the group consisting of synthetic smectic clays, silicates, fluorosilicates and mixtures thereof.

In particularly preferred embodiments of the present invention, the pharmaceutically acceptable carrier further comprises at least one diluent. Suitable diluents are known and available to those skilled in the art. Illustrative examples of suitable diluents include, but are not limited to, spray-dried or anhydrous lactose, sucrose, dextrose, mannitol, sorbitol, starch (e.g. starch 1500), cellulose (e.g. microcrystalline cellulose; Avicel), dihydrated or anhydrous dibasic calcium phosphate (available commercially under the registered trademark Emcompress from Mendell or A-Tab and Di-Tab from Rhone-Poulenc, Inc., Monmouth Junction, N.J.), calcium carbonate, calcium sulfate, and others as known in the art. An appropriate amount of diluent may be determined empirically by one skilled in the art.

According to more particularly preferred embodiments of the present invention, the diluent is a mixture of lactose and microcrystalline cellulose. Preferably, the lactose in such a mixture is lactose monohydrate and more preferably it is Fast-Flo lactose. According to such embodiments of the present invention, the lactose and microcrystalline cellulose are present in a suitable ratio, preferably about 1:2.

In other particularly preferred embodiments of the present invention, the pharmaceutically acceptable carrier further comprises at least one binder. Suitable binders are known and available to those skilled in the art. Illustrative examples of suitable binders include, but are not limited to, acacia, cellulose derivatives (such as methylcellulose and carboxymethylcellulose, hydroxypropylmethylcellulose, hydroxypropylcellulose, hydroxyethylcellulose), gelatin, glucose, dextrose, xylitol, polymethacrylates, polyvinylpyrrolidone, starch paste, sucrose, sorbitol, pregelatinized starch, gum tragacanth, alginic acids and salts thereof such as sodium alginate, magnesium aluminum silicate, polyethylene glycol, guar gum, bentonites, and the like. An appropriate amount of binder may be determined empirically by one skilled in the art.

In other particularly preferred embodiments of the present invention, the pharmaceutically acceptable carrier further comprises at least one disintegrant. Suitable disintegrants are known and available to those skilled in the art. Illustrative examples of suitable disintegrants include, but are not limited to, starch, pregelatinized starch, sodium starch glycolate, sodium carboxymethylcellulose, crosslinked sodium carboxymethylcellulose (sodium croscarmellose; crosslinked starch available under the registered trademark Ac-Di-Sol from FMC Corp., Philadelphia, Pa.), clays (e.g. magnesium aluminum silicate), microcrystalline cellulose (of the type available under the registered trademark Avicel from FMC Corp. or the registered trademark Emcocel from Mendell Corp., Carmel, N.Y.), alginates, gums, surfactants, effervescent mixtures, hydrous aluminum silicate, cross-linked polyvinylpyrrolidone (available commercially under the registered trademark PVP-XL from International Specialty Products, Inc.), and others as known in the art. Preferably, the pharmaceutical composition contains about 1-25% disintegrant, more preferably about 3-15% disintegrant, based on the total weight of the composition.

n other particularly preferred embodiments of the present invention, the pharmaceutically acceptable carrier further comprises at least one lubricant. Suitable lubricants are known and available to those skilled in the art. Illustrative examples of suitable lubricants include, but are not limited to, magnesium stearate, stearic acid, glycerylbehaptate, polyethylene glycol, ethylene oxide polymers (for example, available under the registered trademark Carbowax from Union Carbide, Inc., Danbury, Conn.), sodium lauryl sulfate, magnesium lauryl sulfate, sodium oleate, sodium stearyl fumarate, DL-leucine, colloidal silica, and others as known in the art. Preferred lubricants are magnesium stearate, and mixtures of magnesium stearate with sodium lauryl sulfate. Most preferably, the lubricant is magnesium stearate. Lubricants generally comprise 0.5 to 7.0% of the total weight of the pharmaceutical composition.

The pharmaceutically acceptable carrier may also comprise a variety of conventional excipients, depending on the exact formulation, such as flavorings, buffers, colors, sweetening agents and glidants.

Flavors incorporated in the composition may be chosen from synthetic flavor oils and flavoring aromatics and/or natural oils, extracts from plants leaves, flowers, fruits, and so forth and combinations thereof. These may include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil. Also useful as flavors are vanilla, citrus oil, including lemon, orange, grape, lime and grapefruit, and fruit essences, including apple, banana, pear, peach, strawberry, raspberry, cherry, plum, pineapple, apricot, and so forth. The amount of flavoring may depend on a number of factors including the organoleptic effect desired. Generally, the flavoring will be present in an amount of from 0.5 to about 3.0% by weight based on the total weight of the composition, when a flavor is used.

Coloring agents may include titanium dioxide and/or dyes suitable for food such as those known as F. D. & C, dyes and natural coloring agents such as grape skin extract, beet red powder, beta carotene, annato, carmine, turmeric, paprika, and so forth. A coloring agent is an optional ingredient in the compositions of this invention, but when used will generally be present in an amount up to about 3.5% based on the total weight of the composition.

Suitable dosages of the active ingredient can easily be determined empirically by one having ordinary skill in the art. Doses may be varied according to the age, body weight, severity of the disorder or disease being treated and other factors known to those skilled in the art. Preferably, galantamine is employed in total dosage of up to about 2000 mg per day, more preferably about 5 to about 1000 mg per day and most preferably about 100 to about 600 mg per day.

Preferably, the prolonged release pharmaceutical composition of the present invention is a solid dosage form. Illustrative examples of suitable solid dosage forms include, but are not limited to, tablets, capsules, caplets and dragees. Such solid dosage forms may be prepared by any of the methods and techniques known and available to those skilled in the art.

For example, tablets may be prepared by wet granulation and subsequent compression. A mixture containing the active ingredient and at least one diluent, and optionally a disintegrating agent, is granulated together with an aqueous, ethanolic or aqueous-ethanolic solution of one or more binding agents in appropriate equipment, then the granulate is dried. Thereafter, other preservative, surface acting, dispersing, disintegrating, gliding and anti-adhesive additives can be mixed to the dried granulate and the mixture can be compressed into a tablet.

Tablets may also be prepared by the direct compression of a mixture containing the active ingredient together with the needed additives.

If desired, the tablets may be transformed to dragees by using protective, flavoring and dyeing agents such as sugar, cellulose derivatives (methyl- or ethylcellulose or sodium carboxymethylcellulose), polyvinylpyrrolidone, calcium phosphate, calcium carbonate, food dyes, aromatizing agents, iron oxide pigments and the like which are commonly used in the pharmaceutical industry.

For the preparation of capsules or caplets, a mixture of the active ingredient and the desired additives may be filled into a capsule, such as a hard or soft gelatin capsule.

A second preferred embodiment of the present invention is directed to a method for treating or preventing memory impairment in a mammal in need thereof comprising administering to said mammal a pharmaceutical composition comprising: (a) a therapeutically effective amount of galantamine or a pharmaceutically acceptable salt thereof; and (b) a pharmaceutically acceptable carrier, wherein the pharmaceutical composition provides release of not more than about 30% of the galantamine or pharmaceutically acceptable salt thereof in about 1 hour and at least about 80% of the galantamine or salt thereof in about ten hours as measured in USP buffer pH 6.8 at 37° C. using USP Dissolution Apparatus 2 at 50 rpm.

According to a particularly preferred embodiment of the present invention, the memory impairment being treated is memory impairment dementia, such as that associated with Alzheimer's disease. Other forms of memory impairment dementia include, but are not limited to, vascular dementia, Lewy body disease, autism, metal retardation, bipolar disorder, attention deficit hyperactivity disorder, substance abuse, extreme aggression, nicotine cessation and withdrawal.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

### EXAMPLES

### Example 1

### Galantamine HBr Prolonged Release Tablets (8 mg)

| Ingredient | % | mg |
|---|---|---|
| Galantamine HBr | 2.16 | 008.04 |
| Xanthum gum | 19.35 | 071.98 |
| Locust bean gum | 58.06 | 215.98 |
| Microcrystalline cellulose (Avicel PH-101) | 13.51 | 050.26 |
| Lactose monohydrate (Fast-Flo 316) | 6.76 | 025.15 |
| Magnesium stearate | 0.16 | 000.59 |
| Total | 100 | 372.00 mg |

Tablet weight: 372 mg
Tooling: R 0.3125″

Manufacturing procedure:

1. Each ingredient was weighed.

2. The ingredients were placed into a planetary mixer and mixed for one minute.

3. The mixed ingredients were granulated using a planetary mixer with deionized water to suitable granulation.

4. The granules were dried at 60□ C. overnight.

5. The dried granules were screened through a US standard sieve 25 mesh screen.

6. Oversized granules were milled and then screened through a US standard 25 mesh screen.

7. Magnesium stearate was blended with the screened granules.

8. The blended material was compressed into tablets using a rotary tablet press.

### Example 2

### Galantamine HBr Prolonged Release Tablets

| Ingredient | % | mg |
|---|---|---|
| Galantamine HBr | 1.87 | 008.01 |
| Xanthum gum | 16.80 | 072.00 |
| Locust bean gum | 50.40 | 216.01 |
| Povidone (Plasdone k-29/32) | 13.20 | 056.58 |
| Microcrystalline cellulose (Avicel PH-101) | 11.73 | 050.27 |
| Lactose monohydrate (Fast-Flo 316) | 5.86 | 025.12 |
| Magnesium stearate | 0.14 | 000.61 |
| Total | 100.00 | 428.6 mg |

Tablet weight: 428.6 mg
Tooling: R 0.3125″

Manufacturing procedure:

1. Each ingredient was weighed.

2. The ingredients were placed into a planetary mixer and mixed for one minute.

3. The mixed ingredients were granulated using a planetary mixer with deionized water to suitable granulation.

4. The granules were dried at 60□ C. overnight.

5. The dried granules were screened through a US standard sieve 25 mesh screen.

6. Oversized granules were milled and then screened through a US standard 25 mesh screen.

7. Magnesium stearate was blended with the screened granules.

8. The blended material was compressed into tablets using a rotary tablet press.

### Example 3

### Galantamine HBr Prolonged Release Tablets

| Ingredient | % | mg |
|---|---|---|
| Galantamine HBr | 1.87 | 008.01 |
| Xanthum gum | 16.8 | 072.00 |
| Locust bean gum | 50.40 | 216.01 |
| Povidone (Plasdone K-29/32) | 13.20 | 056.58 |
| Microcrystalline cellulose (Avicel PH-101) | 11.73 | 050.27 |
| Lactose monohydrate (Fast-Flo 316) | 5.86 | 025.12 |
| Magnesium stearate | 0.14 | 000.61 |
| Total | 100 | 428.60 mg |

Tablet weight: 428.60 mg
Tooling: R 0.3125″

Manufacturing procedure:

1. Each ingredient was weighed.

2. The ingredients were placed into a planetary mixer and mixed for one minute.

3. The mixed ingredients were granulated using a planetary mixer with deionized water to suitable granulation.

4. The granules were dried at 60□ C. overnight.

5. The dried granules were screened through a US standard sieve 25 mesh screen.

6. Oversized granules were milled and then screened through a US standard 25 mesh screen.

7. Magnesium stearate was blended with the screened granules.

8. The blended material was compressed into tablets using a rotary tablet press.

### Example 4

### Dissolution Profiles

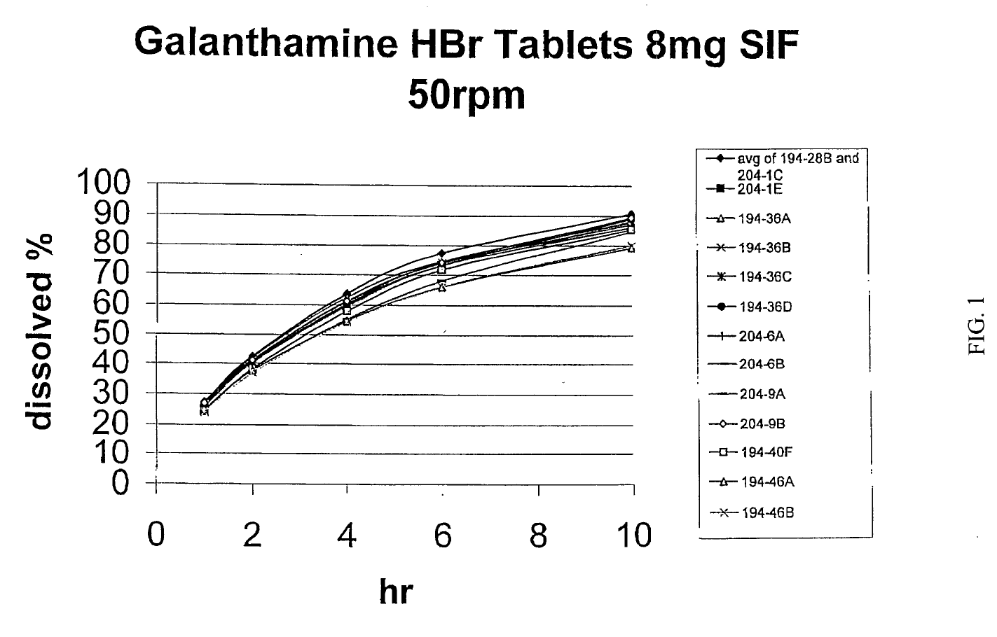

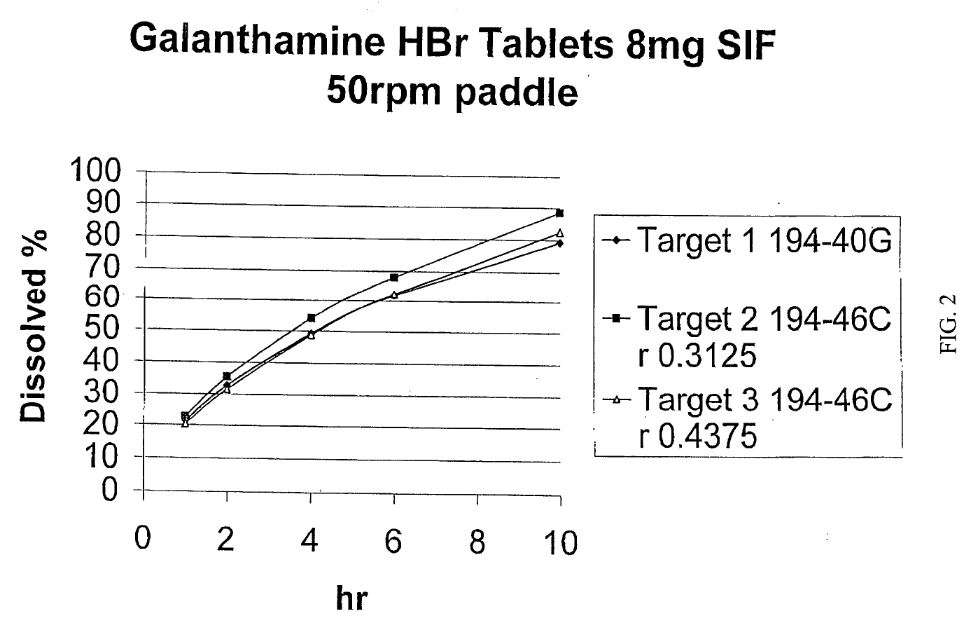

The dissolution profiles of the tablets prepared in Examples 2 and 3 were measured in USP buffer pH 6.8 at 37° C. using USP Dissolution Apparatus 2 at 50 rpm. The results are shown in FIGS. 1 and 2.

The foregoing embodiments and advantages are merely exemplary and are not to be construed as limiting the present invention. The present teaching can be readily applied to other types of apparatuses. The description of the present invention is intended to be illustrative, and not to limit the scope of the claims. Many alternatives, modifications, and variations will be apparent to those skilled in the art. In the claims, means-plus-function clauses are intended to cover the structures described herein as performing the recited function and not only structural equivalents but also equivalent structures. [For example, although a nail and a screw may not be structural equivalents in that a nail employs a cylindrical surface to secure wooden parts together, whereas a screw employs a helical surface, in the environment of fastening wooden parts, a nail and a screw may be equivalent structures. For the italic text, please type in a similar statement relevant to the application.]

What is claimed is:

1. A once a day pharmaceutical composition comprising a therapeutically effective amount of galantamine or a pharmaceutically acceptable salt thereof and a pharmaceutically acceptable carrier, wherein said pharmaceutical composition provides release of at least about 5% of said therapeutically effective amount of galantamine or pharmaceutically acceptable salt thereof in about 2 hour and at least about 50% of said therapeutically effective amount of galantamine or salt thereof in about ten hours as measured in USP buffer pH 6.8 at 37° C. using USP Dissolution Apparatus 2 at 50 rpm.

2. The once a day pharmaceutical composition of claim 1, wherein said pharmaceutically acceptable salt of galantamine is galantamine hydrobromide.

3. The once a day pharmaceutical composition of claim 1, wherein said pharmaceutically acceptable carrier comprises at least one pharmaceutically acceptable gelling agent.

4. The once a day pharmaceutical composition of claim 3, wherein said pharmaceutically acceptable gelling agent is selected from the group consisting of pharmaceutically acceptable water soluble organic gums, pharmaceutically acceptable natural clays, pharmaceutically acceptable synthetic clays and mixtures thereof.

5. The once a day pharmaceutical composition of claim 4, wherein said pharmaceutically acceptable water soluble organic gums are selected from the group consisting of alginates, alkyl celluloses, hydroxyalkylcelluloses, alkyl hydroxyalkylcelluloses, carboxyalkylcelluloses, carrageenan, guar gum, agar, gum arabic, gum ghatti, gum karaya, gum tragacanth, locust bean gum, pectins, polyacrylamide, polyacrylic acid, polyethylene glycol, poly(ethylene oxide), polyvinyl alcohol, polyvinylpyrrolidone, starch, tamarind gum, xanthum gum and mixtures thereof.

6. The once a day pharmaceutical composition of claim 4, wherein said pharmaceutically acceptable natural clays are selected from the group consisting of kaolins, serpentines, smectites (montmorillonites), bentonites, illites, glauconite, chlorites, vermiculites, mixed-layer clays, attapulgite, saponite, sepiolite and mixtures thereof.

7. The once a day pharmaceutical composition of claim 4, wherein said pharmaceutically acceptable synthetic clays are selected from the group consisting of synthetic smectic clays, silicates, fluorosilicates and mixtures thereof.

8. The once a day pharmaceutical composition of claim 4, wherein said pharmaceutically acceptable gelling agent is a mixture of xanthum gum and locust bean gum.

9. The once a day pharmaceutical composition of claim 3, wherein said pharmaceutically acceptable carrier further comprises at least one diluent.

10. The once a day pharmaceutical composition of claim 9, wherein said diluent is selected from the group consisting of lactose, sucrose, dextrose, mannitol, sorbitol, starch, microcrystalline cellulose, dibasic calcium phosphate, calcium carbonate and calcium sulfate.

11. The once a day pharmaceutical composition of claim 9, wherein said diluent comprises a mixture of lactose and microcystalline cellulose.

12. The once a day pharmaceutical composition of claim 11, wherein said lactose is spray-dried (Fast-Flo) lactose.

13. The once a day pharmaceutical composition of claim 11, wherein the ratio of said lactose to said microcrystalline cellulose is about 1:2.

14. The once a day pharmaceutical composition of claim 3, wherein said pharmaceutically acceptable carrier further comprises at least one binder.

15. The once a day pharmaceutical composition of claim 14, wherein said binder comprises polyvinylpyrrolidine.

16. The once a day pharmaceutical composition of claim 3, wherein said pharmaceutically acceptable carrier further comprises at least one lubricant.

17. The once a day pharmaceutical composition of claim 16, wherein said lubricant comprises magnesium stearate.

18. A method of preventing cognitive or other central nervous system impairment in a human, comprising administering to a human in need thereof a once a day pharmaceutical composition of claim 1.

19. A once a day pharmaceutical composition comprising a therapeutically effective amount of galantamine or a pharmaceutically acceptable salt thereof and a pharmaceutically acceptable carrier, wherein said pharmaceutical composition provides release of not more than about 5% of said therapeutically effective amount of galantamine or pharmaceutically acceptable salt thereof in about 2 hours, at least about 25% of said therapeutically effective amount of galantamine or salt thereof in about six hours, and at least about 50% of said therapeutically effective amount of galantamine or salt thereof in about ten hours as measured in USP buffer pH 6.8 at 37° C. using USP Dissolution Apparatus 2 at 50 rpm.

20. A once a day pharmaceutical composition comprising a therapeutically effective amount of galantamine or a pharmaceutically acceptable salt thereof and a pharmaceutically acceptable carrier, wherein said pharmaceutical composition provides release of at least about 8% of said therapeutically effective amount of galantamine or pharmaceutically acceptable salt thereof in about 2 hours, at least about 30% of said therapeutically effective amount of galantamine or salt thereof in about six hours, and at least about 60% of said therapeutically effective amount of galantamine or salt thereof in about ten hours as measured in USP buffer pH 6.8 at 37° C. using USP Dissolution Apparatus 2 at 50 rpm.

21. A once a day pharmaceutical composition comprising a therapeutically effective amount of galantamine or a pharmaceutically acceptable salt thereof and a pharmaceutically acceptable carrier, wherein said pharmaceutical composition provides release of at least about 10% of said therapeutically effective amount of galantamine or pharmaceutically acceptable salt thereof in about 2 hours, at least about 35% of said therapeutically effective amount of galantamine or salt thereof in about six hours, and at least about 65% of said therapeutically effective amount of galantamine or salt thereof in about ten hours as measured in USP buffer pH 6.8 at 37° C. using USP Dissolution Apparatus 2 at 50 rpm.

22. A once a day pharmaceutical composition according to claim 1, 19, 20 or 21 wherein less than about 90% of the active agent is released after about 10 hours.

23. A once a day pharmaceutical composition according to claim 1, 19, 20 or 21 wherein the mean plasma galantamine peak concentration to trough concentration ratio is less than 2 at steady state.

24. A method of using a once a day pharmaceutical composition according to claim 1, 19, 20 or 21 to prevent cognitive or mental performance impairment in a mammal in need of such treatment.

25. A method according to claim 24 wherein the cognitive or mental impairment is caused by primary or secondary memory impairment, toxic, secondary to medical or psychiatric, Alzheimer's, vascular and other dementias, Attention Deficit Disorder, Fibromyalgia, Chronic Fatigue Syndrome, PTSD, Downs Syndrome, anxiety, depression, apathy and agitation.
